(19) United States
(12) Patent Application Publication
(10) Pub. No.: US 2017/0204157 A1
(43) Pub. Date: Jul. 20, 2017

(54) HUMAN FGFR2C EXTRACELLULAR PROTEIN AS WELL AS CODING GENE AND APPLICATION THEREOF

(71) Applicants: Jinan University, Guangzhou City (CN); GUANGZHOU SHENGLU BIOTECH LIMITED COMPANY, Guangzhou City (CN)

(72) Inventors: Ju Wang, Guangzhou City (CN); Anan Chen, Guangzhou City (CN)

(21) Appl. No.: 15/327,881

(22) PCT Filed: Jul. 21, 2015
(86) PCT No.: PCT/CN2015/084692

§ 371 Date: Feb. 21, 2017

(30) Foreign Application Priority Data
Jul. 21, 2014 (CN) 2014103479380

(51) Int. Cl. C07K 14/71

(52) U.S. Cl. CPC C07K 14/71; C07K 2319/30; A61K 38/00

(57) ABSTRACT

The present invention provides an isolated FGFR2c extracellular protein. The extracellular protein comprises a wild type or S252W mutant human FGFR2c extracellular 146-356 site amino acid. The present invention also provides a nucleic acid for coding the extracellular protein, a vector and a host cell as well as a corresponding pharmaceutical composition for treating tumors.

The present patent application claims the priority of Chinese Patent Application No. CN201410347938.0 (Title: FGFR2c extracellular domain analog, and coding gene and application thereof, filing date: Jul. 21, 2014), which is incorporated herein in its entirety by reference.

### TECHNICAL FIELD

The present invention relates to the field of genetic engineering, in particular to the human FGFR2c extracellular fragment of amino acid positions 146-356 and its coding gene and application.

### BACKGROUND ART

EGFR is the expression product of the proto-oncogene c-erbB1 and is a member of the epidermal growth factor receptor (HER) family. The family includes HER1 (erbB1, EGFR), HER2 (erbB2, NEU), HER3 (erbB3) and HER4 (erbB4). The HER family plays an important role in regulating physiological processes of cells. The EGFR signaling pathway plays an important role on physiological processes such as cell growth, proliferation and differentiation. Loss of the function of protein tyrosine kinases such as EGFR and abnormal activity or cellular localization of key factors in its associated signaling pathways will lead to the development of malignant tumors, diabetes, immune deficiencies and cardiovascular diseases. EGFR is also an important molecular target for the treatment of malignant tumors.

However, inhibition of EGF signaling often leads to activation of FGF signaling, and induces drug resistance. Therefore, if a protein molecule which inhibits EGF signaling but do not activates FGF signaling can be found, better effect in the treatment of malignant tumors can be expected.

### SUMMARY OF THE INVENTION

The object of the present invention is to provide a protein molecule which can inhibit EGF signaling but does not activate FGF signaling.

In order to solve the above technical problem existing in the prior art, the inventors conducted intensive study and found that a protein comprising human FGFR2c extracellular fragment of amino acid positions 146-356 and its variants can inhibit EGF signaling, but do not activate FGF signaling. Thus, the present invention is completed.

That is, the present invention comprises the following embodiments.

1. An isolated protein comprising the following amino acid sequence:

i) the amino acid sequence as set forth in SEQ ID NO: 1 or 2.

2. An isolated protein comprising the following amino acid sequence and having the function of inhibiting EGF signaling but not activating FGF signaling:

ii) an amino acid sequence obtained from the amino acid sequence as set forth in SEQ ID NO: 1 or 2 by deleting, substituting, inserting, and/or adding one or more amino acids;

iii) an amino acid sequence having a homology of 80% or more with the amino acid sequence as set forth in SEQ ID NO: 1 or 2; or

vi) an amino acid sequence encoded by a nucleic acid which hybridizes under stringent conditions to the complementary strand of the nucleic acid encoding the amino acid sequence as set forth in SEQ ID NO: 1 or 2.

3. The isolated protein according to embodiment 2, wherein the isolated protein has a human origin.

4. An isolated nucleic acid encoding the isolated protein of any one of embodiments 1 to 3.

5. A vector comprising the nucleic acid of embodiment 4.

6. A host cell comprising the vector of embodiment 5.

7. The host cell according to embodiment 6, wherein said host cell is any one of CHO cells, E. coli cells, insect cells, and yeast cells.

8. A fusion protein of the isolated protein of any one of embodiments 1 to 3 fused with another polypeptide.

9. The fusion protein according to embodiment 8, wherein the isolated protein of any one of embodiments 1 to 3 is fused with an epitope tag sequence of a human immunoglobulin or the FPc portion of a human immunoglobulin.

10. Use of the isolate protein of any one of embodiments 1 to 3, the nucleic acid of embodiment 4, the vector of embodiment 5, the host cell of embodiment 6 or 7, or the fusion protein of embodiment 8 or 9 in the manufacture of a medicament for treating malignant tumors.

11. A pharmaceutical composition for treating malignant tumors, which comprises the isolate protein of any one of embodiments 1 to 3, the nucleic acid of embodiment 4, the vector of embodiment 5, the host cell of embodiment 6 or 7, or the fusion protein of embodiment 8 or 9 as the active ingredient, and a pharmaceutically acceptable carrier.

### DESCRIPTION OF THE DRAWINGS

FIG. 1 shows the SDS-PAGE results of human FGFR2c extracellular fragment of amino acid positions 146-356 (sFGFR2c) as described in Example 1; wherein Lane M is protein marker, Lane 1 is induced wild type (wsFGFR2c), Lane 2 is uninduced wild type, Lane 3 is induced S252W mutant (msFGFR2c), and Lane 4 is uninduced S252W mutant.

FIG. 2 shows the Western blot protein hybridization results of sFGFR2c as described in Example 1; wherein Lane 1 is wild type and Lane 2 is S252W mutant.

FIG. 3 shows the comparison of the stability and the effect of inhibiting tumor cells of sFGFR2c (146-356), sFGFR2c (aa. 147-366) and sFGFR2c (aa. 151-377) as described in Example 7; wherein Panel A is an electrophoretogram showing the stability of sFGFR2c, Panel B is an electrophoretogram showing the stability of sFGFR2c (aa. 147-366) and Panel C is an electrophoretogram showing the stability of sFGFR2c (aa. 151-377).

FIG. 4 shows the co-IP results demonstrating the capacity of sFGFR2c to bind to EGFR as described in Example 8; wherein, wsFGFR2c is wild-type human FGFR2c extracellular fragment 146-356, and msFGFR2c is S252W mutant FGFR2c extracellular fragment 146-356.

FIG. 5 shows the CCK8 results demonstrating the effect of sFGFR2c on proliferation of DU145 cells as described in Example 8; wherein wsFGFR2c is wild type human FGFR2c extracellular fragment 146-356, msFGFR2c is S252W mutant human FGFR2c extracellular fragment 146-356; and wherein ▴ represents P <0.01 as compared to the blank control group, and ★ represents P <0.01 as compared to the EGF induction alone group.

FIG. 6 shows the protein blotting results demonstrating the effect of human FGFR2c extracellular fragment 146-356 on EGFR/ERK signaling pathway; wherein wsFGFR2c is wild type human FGFR2c extracellular fragment 146-356, and msFGFR2c is S252W mutant human FGFR2c extracellular fragment 146-356.

FIG. 7 shows the experimental results of inhibition of FGFRs and ERK phosphorylation by sFGFR2c as detected by Western blot.

FIG. 8 shows the experimental results demonstrating the interaction of FGFR2c extracellular fragment 146-356 with EGFR as detected by isothermal titration calorimetry (iTC).

### SPECIFIC EMBODIMENTS

1. Isolated Proteins

In one aspect, the present invention provides an isolated protein comprising the following amino acid sequence:

i) the amino acid sequence as set forth in SEQ ID NO: 1 or 2.

In the present specification, the term “comprising” preferably means “consisting of”. The protein consisting of the amino acid sequence as set forth in SEQ ID NO: 1 is the human FGFR2c extracellular fragment of amino acid positions 146-356 (wsFGFR2c), and the protein consisting of the amino acid sequence as set forth in SEQ ID NO: 2 is the protein obtained from the protein consisting of the amino acid sequence as set forth in SEQ ID NO: 1 by mutating serine (S) at the position corresponding to position 252 of the full length wild type human FGFR2c extracellular fragment to tryptophan (W) (msFGFR2c). In the present specification, proteins consisting of the amino acid sequence as set forth in SEQ ID NO: 1 or 2 are collectively referred to as human FGFR2c extracellular fragment 146-356.

SEQ ID NO: 1
Asn Lys Arg Ala Pro Tyr Trp Thr Asn Thr Glu Lys Met Glu Lys Arg
Leu His Ala Val Pro Ala Ala Asn Thr Val Lys Phe Arg Cys Pro Ala
Gly Gly Asn Pro Met Pro Thr Met Arg Trp Leu Lys Asn Gly Lys Glu
Phe Lys Gln Glu His Arg Ile Gly Gly Tyr Lys Val Arg Asn Gln His
Trp Ser Leu Ile Met Glu Ser Val Val Pro Ser Asp Lys Gly Asn Tyr
Thr Cys Val Val Glu Asn Glu Tyr Gly Ser Ile Asn His Thr Tyr His
Leu Asp Val Val Glu Arg Ser Pro His Arg Pro Ile Leu Gln Ala Gly
Leu Pro Ala Asn Ala Ser Thr Val Val Gly Gly Asp Val Glu Phe Val
Cys Lys Val Tyr Ser Asp Ala Gln Pro His Ile Gln Trp Ile Lys His
Val Glu Lys Asn Gly Ser Lys Tyr Gly Pro Asp Gly Leu Pro Tyr Leu
Lys Val Leu Lys Ala Ala Gly Val Asn Thr Thr Asp Lys Glu Ile Glu
Val Leu Tyr Ile Arg Asn Val Thr Phe Glu Asp Ala Gly Glu Tyr Thr
Cys Leu Ala Gly Asn Ser Ile Gly Ile Ser Phe His Ser Ala Trp Leu
Thr Val Leu
SEQ ID NO: 2
Asn Lys Arg Ala Pro Tyr Trp Thr Asn Thr Glu Lys Met Glu Lys Arg
Leu His Ala Val Pro Ala Ala Asn Thr Val Lys Phe Arg Cys Pro Ala
Gly Gly Asn Pro Met Pro Thr Met Arg Trp Leu Lys Asn Gly Lys Glu
Phe Lys Gln Glu His Arg Ile Gly Gly Tyr Lys Val Arg Asn Gln His
Trp Ser Leu Ile Met Glu Ser Val Val Pro Ser Asp Lys Gly Asn Tyr
Thr Cys Val Val Glu Asn Glu Tyr Gly Ser Ile Asn His Thr Tyr His
Leu Asp Val Val Glu Arg Ser Pro His Arg Pro Ile Leu Gln Ala Gly
Leu Pro Ala Asn Ala Ser Thr Val Val Gly Gly Asp Val Glu Phe Val
Cys Lys Val Tyr Ser Asp Ala Gln Pro His Ile Gln Trp Ile Lys His
Val Glu Lys Asn Gly Ser Lys Tyr Gly Pro Asp Gly Leu Pro Tyr Leu
Lys Val Leu Lys Ala Ala Gly Val Asn Thr Thr Asp Lys Glu Ile Glu
Val Leu Tyr Ile Arg Asn Val Thr Phe Glu Asp Ala Gly Glu Tyr Thr
Cys Leu Ala Gly Asn Ser Ile Gly Ile Ser Phe His Ser Ala Trp Leu
Thr Val Leu

Fibroblast growth factors (FGF) receptor (FGF receptor, FGFR) is a membrane receptor. Its extracellular portion can bind to specific ligands, and its intracellular portion has tyrosine kinase activity. When the extracellular portion binds to a ligand, dimerization and phosphorylation of the receptor can be activated, resulting in the activation of downstream signaling, thereby regulating the expression of the target genes. FGFR2c extracellular fragment refers to the extracellular portion of FGF receptors of the subtype 2c. It can bind to a ligand, reducing the effective concentration of the ligand, thereby inhibiting FGF signaling. The inventors have verified that the above isolated protein can inhibit EGF signaling, but does not activate (preferably inhibits) FGF signaling.

In another aspect, the present invention provides an isolated protein comprising the following amino acid sequence and having the function of inhibiting EGF signaling, but does not activate FGF signaling:

ii) an amino acid sequence obtained from the amino acid sequence as set forth in SEQ ID NO: 1 or 2 by deleting, substituting, inserting, and/or adding one or more amino acids;

iii) an amino acid sequence having a homology of 80% or more, preferably 85% or more, more preferably 87.8% or more, more preferably 90% or more, more preferably 95% or more, more preferably 96% or more, more preferably 97% or more, more preferably 98% or more, more preferably 99% or more, more preferably 99.5% or more with the amino acid sequence as set forth in SEQ ID NO: 1 or 2; or

vi) an amino acid sequence encoded by a nucleic acid which hybridizes under stringent conditions to the complementary strand of the nucleic acid encoding the amino acid sequence as set forth in SEQ ID NO: 1 or 2.

In the present specification, the above isolated proteins are referred to as variants of human FGFR2c extracellular fragment 146-356.

Preferably, said variants of human FGFR2c extracellular fragment 146-356 have a human origin.

In said variants of human FGFR2c extracellular fragment 146-356, the amino acid substitution can be a conservative substitution, i.e., substitution of a specific amino acid residue with a residue having similar physical and chemical characteristics. Non-limiting examples of conservative substitution include substitutions between amino acid residues containing an aliphatic group (e.g., substitutions of Ile, Val, Leu, or Ala with each other), substitutions between polar residues (e.g., substitutions between Lys and Arg, Glu and Asp, or Gin and Asn), and the like. For example, variants obtained by amino acid deletion, substitution, insertion, and/or addition can be generated by conducting well-known site-directed mutagenesis (see, e.g., Nucleic Acid Research, Vol. 10, No. 20, pp. 6487-6500,1982, which is incorporated herein by reference in its entirety) on DNA encoding the wild type protein or methods of artificially synthesizing proteins. In the present specification, the phrase “one or more amino acids” refers to the number of amino acids that can be deleted, substituted, inserted, and/or added by site-directed mutagenesis or methods of artificial synthesis, e.g., 1 to 20 amino acids, preferably 1 to 15 amino acids, more preferably 1 to 10 amino acids, more preferably 1 to 8 amino acids, more preferably 1 to 2 amino acids, more preferably 1 amino acid.

The homology % between two amino acid sequences can be determined by visual inspection and mathematical calculation. Alternatively. the percentage of homology between two polypeptide sequences can be determined by comparing the sequence information using the GAP computer program available from the University of Wisconsin, Genetics Computer Group (UWGCG), which is based on the algorithm of Needleman, S. B. and Wunsch, C. D. (J. Mol. Bol., 48: 443-453, 1970). Preferably, the default parameters for the GAP program include: (1) blosum 62, the score/matrix described by Henikoff, S. and Henikoff J. G. (Proc. Natl. Acad. Sci. USA, 89: 10915-10919, 1992); (2) a gap penalty of 12; (3) a continuous gap penalty of 4; and (4) a terminal gap penalty of null. Other programs used by those skilled in the art for sequence comparison can also be used. As for the percentage of homology, for example, the BLAST program as described by Altschul et al. (Nucl. Acids Res., 25, pp. 3389-3402, 1997) can be used to compare and determine the sequence information. Said program can be executed on the network at the website of National Center for Biotechnology Information (NCBI) or DNA Data Bank of Japan (DDBJ). At the same sites, various conditions (parameters) for homology search by the BLAST program are described in detail. Although some settings can be appropriately altered, searches are usually carried out with the default values. In addition, the homology % between two amino acid sequences can also be determined using the softwares such as the genetic information processing software GENETYX Ver. 7 (GENETYX Ltd.) or the algorithms such as FASTA. At this time, searches can also be carried out with the default values.

In the present specification, the term “stringent conditions” refers to the conditions under which so-called specific hybrids are formed and non-specific hybrids are not formed. Examples of stringent conditions include such conditions under which DNAs having high homology hybridize with each other, e.g., DNAs which are no less than 80% homologous, preferably no less than 90% homologous, more preferably no less than 95% homologous, still more preferably no less than 97% homologous, particularly preferably no less than 99% homologous hybridize with each other, and DNAs having lower homology than the above ones do not hybridize with each other, or washing conditions of typical Southern hybridization, i.e., conditions corresponding to washing once, preferably 2 or 3 times at the salt concentration and temperature of 1×SSC, 0.1% SDS at 60° C. preferably 0.1×SSC, 0.1% SDS at 60° C., more preferably 0.1×SSC, 0.1% SDS at 68° C.

Whether or not a molecule has the function of “inhibiting EGF signaling, but not activating (preferably inhibiting) FGF signaling” can be determined using the method described in Examples, for example.

2. Isolated Nucleic Acids

In another aspect, the present invention provides an isolated nucleic acid encoding the above human FGFR2c extracellular fragment 146-356 or a variant thereof.

The isolated nucleic acid may be either single-stranded or double-stranded; it may be either DNA or RNA, and may also be a hybrid of DNA and RNA.

The isolated nucleic acid can be prepared by methods of artificial synthesis. For example, it can also be prepared by methods of genetic engineering.

Typically, the amino acid sequence as set forth in SEQ ID NO: 1 or 2 described above can be encoded by the base sequence as set forth in SEQ ID NO: 3 or 4, respectively.

3. Vectors and Host Cells

In another aspect, the present invention provides a vector comprising said nucleic acid.

There is no specific limitation for the type of the vector. It may be one of those conventionally used by those skilled in the art. As a vector, plasmids, phages, animal viruses and the like may be put forward. Preferably, said vector is an expression vector. Said expression vector includes prokaryotic expression vectors and eukaryotic expression vectors, preferably pET3c vectors, pCDNA3.1 vectors, pIRESneo3 vectors, pPICZαA vectors or pFastBac vectors.

In another aspect, the present invention provides a host cell comprising said vector.

There is no specific limitation for the type of the host cell. It may be one of those conventionally used by those skilled in the art. As a host cell, CHO cells, E. coli cells, insect cells, yeast cells and the like may be put forward.

4. Fusion Proteins

In another aspect, the present invention provides a fusion protein of the above human FGFR2c extracellular fragment 146-356 or variants thereof with another polypeptide. Preferably, said another polypeptide is an epitope tag sequence of a human immunoglobulin or the Fc portion of a human immunoglobulin.

Said fusion protein can be prepared using conventional methods in the art.

5. Pharmaceutical Compositions and Pharmaceutical Use

In another aspect, the present invention provides a pharmaceutical composition, which comprises the above human FGFR2c extracellular fragment 146-356 or variant thereof, the above nucleic acid, the above vector, the above host cell, or the above fusion protein as the active ingredient and further comprises a pharmaceutically acceptable carrier. The pharmaceutical compositions of the present invention can be used in the treatment of malignant tumors. Accordingly, in another aspect, the present invention provides use of the above human FGFR2c extracellular fragment 146-356 or variant thereof, the above nucleic acid, the above vector, the above host cell, or the above fusion protein in the manufacture of a medicament for the treatment of malignant tumors.

As a malignant tumor, prostate cancer, oral cancer, cancer of Nasal mucosa, trachea cancer, bronchus cancer, lung cancer, esophagus cancer, stomach cancer or gastric cancer, large intestine cancer, small intestine cancer, liver cancer or hepatic cancer, bile duct cancer, gallbladder cancer, pancreatic cancer, kidney cancer or renal cancer, bladder cancer, urethral cancer, testis cancer, ovarian cancer, thyroid cancer, adrenal cancer, thymus cancer, prostate cancer, breast cancer, tonsil cancer, and the like may be put forward. As a pharmaceutically acceptable carrier, particularly, sterile water, physiological saline, vegetable oils, emulsifiers, suspending agents, surfactants, stabilizers, flavoring agents, excipients, vehicles, preservatives, binding agents, and the like may be put forward.

### EXAMPLES

The present invention will be further described in detail with reference to the following examples and the accompanying drawings, but the embodiments of the present invention are not limited thereto.

For those experimental methods in the following Examples for which the specific conditions are not indicated, they are generally conducted in accordance with conventional conditions, e.g., the conditions described in Sambrook et al., Molecular Cloning: A Laboratory Manual (New York: Cold Spring Harbor Laboratory Press, 1989), or according to the conditions recommended by the manufacturers. The various commonly used chemical reagents used in Examples are all commercially available products. The cell lines and vectors used are all commercially available products. The primers having a designation starting with F generally are upstream primers, and the primers having a designation starting with R generally are downstream primers.

### Example 1. Expression of the Genes of Wild Type and Mutant FGFR2c Extracellular Fragment 146-356 Polypeptides in Escherichia coli

This example describes the process of preparing wild type wsFGFR2c and S252W mutant msFGFR2c.

I. Preparation of the Gene of FGFR2c Extracellular Fragment 146-356 Polypeptide

1. Total RNA for wild type FGFR2c extracellular fragment was extracted from human placenta tissue (fresh placental tissue was obtained at a hospital in Guangdong with consent from the puerpera) using the Trizol method, and a cDNA library was established.

(1) Extracting mRNA using the Trizol method: extraction was conducted using Trizol reagent. The specific steps can be found in the literature “Prokaryotic expression, in vitro renaturation and activity study of mutant βFGFR2IIIc extracellular fragment”, Zhicheng Zhang, Jinan University Master Theses, 2007: 46-47”.

(2) Obtaining cDNA by RT-PCR:

The amount of the individual components of reverse transcription PCR was calculated. The total RNA prepared from the sample in step (1) and DEPC treated water were first added into a PCR tube. The RNA was pre-denaturated on a PCR instrument at 65° C. for 10 min. After pre-denaturation, the tube was immediately plunged into ice. Then other components of the reverse transcription reaction were added, a reverse transcription reaction was conducted and thereby cDNA was obtained. The details of the reaction:

| a. Pre-denaturation | total RNA | 1 | μg |
|---|---|---|---|
| | DEPC water | supplemented to 20 μL | |
| b. reverse transcription | Oligo dT | 1 | μL |
| | 10 mM dNTP | 2 | μL |
| | HRP RNase inhibitor | 0.5 | μL |
| | 5x RT buffer | 4 | μL |
| | reverse transcriptase Ace | 1 | μL |
| | Total volume: | 20 | μL |

Reverse transcription PCR program: 30° C., 10 min; 42° C. I hr; 70° C., 10 min.

2. Design of Primers

The primers used in the present invention were all synthesized at Beijing Liuhe BGI Technology Co., Ltd.

The restriction endonucleases used in the present invention were all purchased from TaKaRa.

The sequence of the primers for the wild type wsFGFR2c gene are as follows:

F1: 5′-CG CATATG AACAAGAGAGCACCATAC-3′; the restriction site of Nde I is underlined;

R1: 5′-AT GGATCC CTATTA CAGAACTGTCAACCATGC-3′; the restriction site of BamH 1 is underlined.

To obtain the gene encoding S252W mutant msFGFR2c, a pair of primers for site-directed mutagenesis were designed as following:

F2: 5′-TTGTGGAGCGATGGCCTCACCGGCCCAT-3′;
R2: 5′-ATGGGCCGGTGAGGCCATCGCTCCACAA-3′.

3. Amplification of the Sequence of the FGFR2c Extracellular Fragment 146-356 Gene:

To amplify the gene encoding wild type FGFR2c extracellular fragment 146-356, PCR was conducted using the primers for wild-type wsFGFR2c (F1 and R1) on the cDNA obtained in step 1 and thereby the gene encoding wild type wsFGFR2c was obtained.

To amplify the gene encoding mutant FGFR2c extracellular fragment 146-356, the gene encoding S252W mutant msFGFR2c was obtained by amplification via the overlap extension PCR method using the gene encoding wild type wsFGFR2c as the template and the PCR primers for the mutant.

The amplification system and reaction conditions for the above PCR were as follows (PrimerSTAR max was purchased from TaKaRa):

PCR reaction system for wild type FGFR2c:

| | |
|---|---|
| PrimerSTAR max | 25 μL |
| F1 | 3 μL |
| R1 | 3 μL |
| template | 4 μL |
| ddH₂O | 15 μL |
| Total volume: | 50 μL |

The gene encoding S252W mutant msFGFR2c was obtained via point mutation via overlapping PCR, which includes two steps.

The system of the first step of the PCR reaction:

System 1:

| | |
|---|---|
| PrimerSTAR max | 25 μL |
| F1 | 3 μL |
| R2 | 3 μL |
| template | 4 μL |
| ddH₂O | 15 μL |
| Total volume | 50 μL |

System 2:

| | |
|---|---|
| PrimerSTAR max | 25 μL |
| R1 | 3 μL |
| F2 | 3 μL |
| template | 4 μL |
| ddH₂O | 15 μL |
| Total volume | 50 μL |

The system of the second step of the PCR reaction:

| | |
|---|---|
| PrimerSTAR max | 25 μL |
| F1 | 3 μL |
| R1 | 3 μL |
| A mixture of the system 1 and the system 2 in equal volume | 4 μL |
| ddH₂O | 15 μL |
| Total volume | 50 μL |

The reaction conditions were: 96° C., 5 min; 31 cycles of 94° C. 15 sec, 60° C., 15 sec, 72° C., 5 sec; 72° C. 10 min.

4. Collection, Purification and Identification Steps:

Electrophoresis was conducted on agarose at the concentration of 1%. The gel was cut. The DNA was recovered using a DNA Gel Recovery Kit (TIANGEN, DP209). The DNA fragment obtained by recovered was detected for the concentration and purity by agarose gel electrophoresis and UV spectrophotometer. The ratio of OD₂₆₀/OD₂₈₀ should be within the range of 1.7-1.9.

II. Construction of the Recombined Vectors of the Genes Encoding Wild-Type and Mutant FGFR2c Extracellular Fragment 146-356 Polypeptides:

1. Constructing Recombined Plasmids by Double Digestion and Ligation Reactions

The PCR amplified sequence and the pET3c vector (from Invitrogen, Co., USA) were subjected to double digestion with Nde I and BamH I, respectively. The digestion reaction conditions: 37° C. water bath for 4 hrs.

After double digestion with Nde I and BamH I, the pET3c plasmid and the FGFR2c gene were ligated using T4 DNA ligase. The reaction system was prepared following the instructions of the T4 DNA ligase. The conditions of the ligation reaction: 16° C. water bath for 12 hrs. The ligated product was obtained.

2. Expression and Identification of the Recombined Plasmid

(1) Transformation of E. coli strain DH5α using the CaCl₂ method. Competent cells of Escherichia coli DH5a were first prepared using CaCl₂. Then the competent cells of E. coli DH5α were transformed with the ligated product obtained in the above step. The specific steps can be found in the literature “Prokaryotic expression, in vitro renaturation and activity study of mutant βFGFR2IIIc extracellular fragment”, Zhicheng Zhang, Jinan University Master Theses, 2007: 46”.

(2) Identification of the Recombined Plasmid Via Double Digestion

Several single colonies were picked and inoculated into 5 ml LB medium containing 100 μg/ml ampicillin (Amp) using a inoculation ring. The tubes were labeled. After culture in a shaker at 37° C. with shaking for 12 hrs, plasmids were extracted (following the manual of OMEGA Plasmide Mini Extraction kit) and identified via double digestion (using Nde I and BamH I double digestion). Monoclones passing the identification via double digestion were selected and sent to Sangon Co. for sequencing. Two recombined plasmids were obtained with the correct sequence, i.e., pET3c-FGFR2c, pET3c-S252W-FGFR2c.

III. Expression of the Gene Encoding FGFR2c Extracellular Fragment 146-356 Polypeptide in E. coli

(1) Following the method of transforming E. coli strain DH5a using CaCl2 as described above, the above two recombined plasmids were transferred into an engineering strain of E. coli BL21 (DE3) (Novagen), respectively, and thereby an expression strain was obtained.

(2) The expression strain of BL21 (DE3) was inoculated into the sterilized LB liquid media containing 0.1% w/v Amp at a ratio of 1:50 based on volume and cultured at 37° C. in a shaker at 200 rpm.

When the bacterial culture reached an OD600 of 0.6 to 0.8, a control group and an induction group were set up. To the induction group, 0.84 M IPTG was added to a final concentration of 0.84 mM. and expression was induced at 37° C. for 3 hrs. The control group received no treatment.

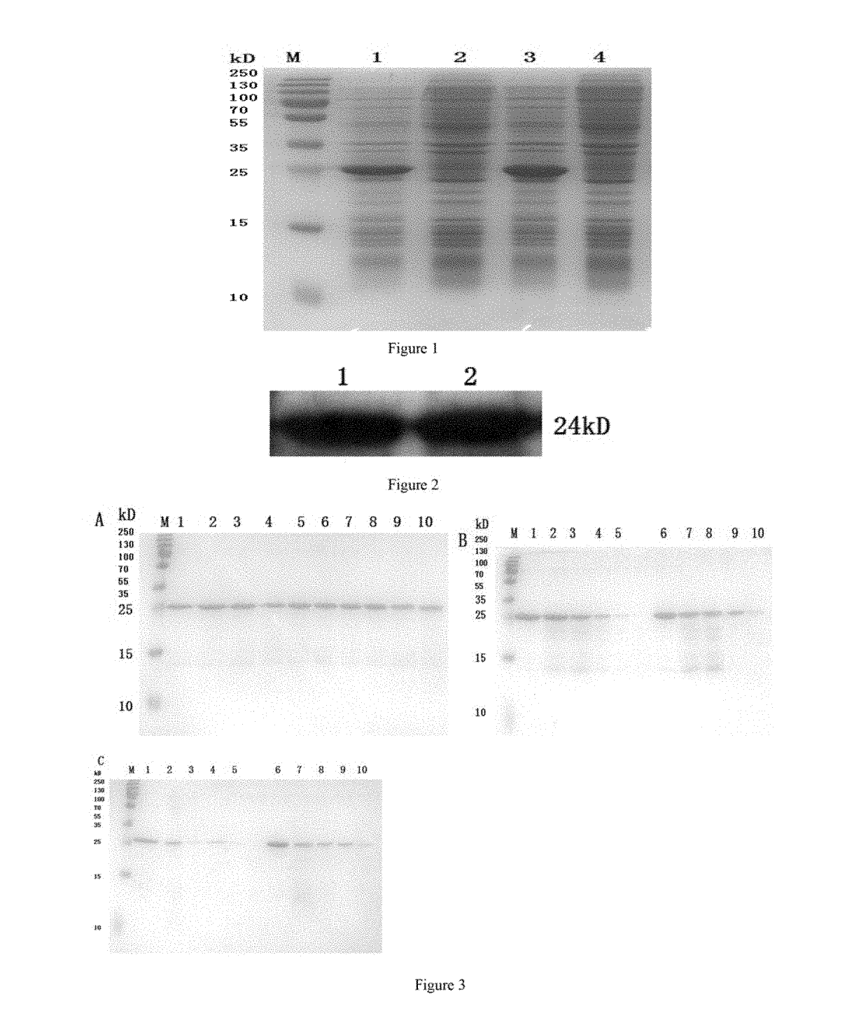

The expression of the protein of interest was identified via SDS-PAGE. The results are shown in FIG. 1. The results demonstrated that IPTG can well induce the expression of FGFR2c extracellular fragment 146-356 by the expression strain of BL21 (DE3).

IV. Collection and Purification of FGFR2c Extracellular Fragment 146-356 Polypeptide

(1) The bacterial cells induced with IPTG were obtained by the method of step III. Then, the pellet was collected by centrifugation at 6000 rpm for 30 min at 4° C.

(2) The bacterial cells were sonicated in the disruption buffer at a ratio of 1 g bacterial cells/8˜10 ml disruption buffer. After disruption was completed, the supernatant was collected by centrifugation at 18,000 rpm for 60 min at 4° C. The conditions for disruption: working, 3 sec; resting, 5 sec; duration, 18 min; amplitude, 65%; disruption buffer, 0.15 M NaCl, 25 mM sodium phosphate buffer (PB, a mixture of sodium monobasic phosphate and sodium dibasic phosphate), 2 mM EDTA, pH=7.5.

(3) In some bacteria for prokaryotic expression, FGFR2c extracellular fragment 146-356 protein is expressed as inclusion bodies. In that case, FGFR2c extracellular fragment 146-356 protein in active form can be obtained from the inclusion bodies by washing and the denaturation-renaturation technology (see Example 1 of the reference patent ZL200710029286.6 for the specific method).

(4) Detected was conducted via Western Blot, using the Bek antibody (c-17) (santa Cruz Biotechnology) as the primary antibody and a rabbit secondary antibody (Cat. No. AS006, Asbio) as the secondary antibody. The results are shown in FIG. 2, which demonstrated that FGFR2c extracellular fragment 146-356 can be specifically recognized by the FGFR antibody.

### Example 2. Expression of FGFR2c Extracellular Fragment 146-356 in Mammalian Cells

This example describes a method of preparating FGFRc extracellular fragment 146-356 polypeptides in potentially glycosylated form by recombinantly expressing the genes encoding wild type wsFGFR2c and S252W mutant msFGFR2c in mammalian cells.

1. Primer Design

F3: 5′-ATAT GGATCC GCCGCCACC ATG AACAAGAGAGCACC
ATAC-3′;
the restriction site of BamH I is underlined;
R3: 5′-GCGCAAGCTT TCATTA CAGAACTGTCAACCATGC-3′;
the restriction site of Hind III is underlined;

2. Vector Construction

The vector pCDNA3.1(−) (purchased from Invitrogen, Co., USA) was used as the expression vector. PCR was conducted using pET3c-FGFR2c and pET3c-S252W-FGFR2c obtained in Example 1 as the template and F3 and R3 as the primers. The reaction system and conditions were same as those in Example 1. The resulting wild type wsFGFR2c gene, S252W mutant msFGFR2c gene were ligated into pCDNA3.1(−), and transformed into competent cells of E. coli DH5a, respectively. The resulting vectors were designated as pCDNA3.1-FGFR2c and pCDNA3.1-FGFR2c-S252W. The specific steps were same as those in Example 1. The endonucleoases for double-digestion were BamH I and Hind III.

3. Transfection of 293 Cells with pCDNA3.1-FGFR2c and pCDNA3.1-FGFR2c-S252W

(1) Plasmids were extracted following the method of endotoxin-free plasmid mini extraction kit (purchased from OMEGA Co.) to obtain the plasmids of pCDNA3.1-FGFR2c and pCDNA3.1-FGFR2c-S252W.

(2) Human embryonic kidney cell, 293T cell (ATCC, CRL-3216) was selected as the host cell. The cells were allowed to grow to confluence in the DMEM medium supplemented with fetal bovine serum (FBS, a final concentration of 10% v/v) at 37° C., 5% CO₂ in an incubator with saturated humidity.

(3) 24 hrs before transfection, 293T cells in log phase growth were trypsinized at a ratio of 0.25% w/v. A suspension was prepared by pipetting the cells in the DMEM medium with neither antibiotic. The cells were plated into a six-well plate, 2×10⁵ cells/well. The cells were allowed to grow to achieve complete adherence and a cell density of 50%-60% at the time of transfection.

(4) Transfection was conducted using the Lipofectamine™ 2000 liposome transfection kit (purchased from Invitrogen Co., USA) with the specific transfection steps as follows.

(i) Dilution of Lipofectamine™ 2000: as calculated for each well of a six-well plate, 5 μL Lipofectamine™ 2000 was added to 250 μL opti-MEM medium for dilution, standing for 5 min.

(ii) Dilution of plasmid to be transfected: as calculated for each well of a six-well plate, 4 pg plasmid to be transfected was added to 250 μL opti-MEM medium for dilution.

(iii) The dilutions obtained in steps (i) and (ii) were mixed at equal volume and allowed to stand for 20 min. To each well, 500 μL mixture was added, and 1.5 ml opti-MEM medium was supplemented. After incubation in an incubator at 37° C., 5% CO₂ for 4-6 hrs, the medium was replaced with the DMEM medium containing 10% FBS v/v. After transfection of 24 hrs, the existing medium was carefully aspirate and replaced with the DMEM complete medium containing 10% (v/v) FBS. Culture was continued.

4. Purification and Identification of FGFR2c Extracellular Fragment 146-356 Protein

The supernatant was collected from the culture by centrifugation at 18,000 rpm for 30 min at 4° C. The protein was purified with the following steps.

A heparin affinity chromatography column (GE 17-0998-01, 50 ml) was washed with 3 column volumes of di-distilled water and balanced with at least 3 column volumes of affinity chromatography column balance solution at a flow rate of 5 ml/min. After the supernatant obtained in step (2) was loaded, the column was washed with 3 column volumes of affinity chromatography balance solution and eluted with heparin elution solution. A single elution peak was collected at the wavelength of 280 nm, to give the wild type wsFGFR2c polypeptide and the S252W mutant msFGFR2c polypeptide. They were stored at −70° C. for subsequent testing.

Affinity chromatography balance solution: 25 mM HEPES, 0.15 M NaCl, pH=7.5;

Heparin elution solution: 25 mM HEPES, 1.5 M NaCl, pH=7.5;

Flow rate: 5 ml/min.

Identification was conducted by Western Blot. The results demonstrated that the FGFR2c extracellular fragment 146-356 could be t specifically recognized by the FGFR antibody.

### Example 3. Expression of FGFR2c Extracellular Fragment 146-356 in CHO Cells

1. Primer Design:

Upstream primer F3: 5′-ATAT GGATCC GCCGCCACC ATG AACAAGAGAGCACCATAC-3′; the restriction site of BamH I is underlined;

Downstream primer R4: 5′-GCGCGAATTC TCATTA CAGAACTGTCAACCATGC-3′; the restriction site of EcoR I is underlined.

2. Vector Construction

The vector pIRESneo3 (purchased from Clontech Co.) was used as the expression vector.

Step 2 of Example 2 was repeated using F3 and R4 as the primers and the restriction endonucleases BamH I and EcoR I. The resulting vectors were designated as pIRESneo3-FGFR2c and pIRESneo3-FGFR2c-S252W.

3. Transfection of CHO-DG44 Cells with pIRESneo3-FGFR2c and pIRESneo3-FGFR2c-S252W

(1) Plasmids were extracted following the endotoxin-free plasmid macro extraction kit to obtain the plasmids of pIRESneo3-FGFR2c and pIRESneo3-FGFR2c-S252W.

(2) Chinese hamster ovary cells, CHO-DG44 cells (Invitrogen Co.) were transfected with the plasmids obtained in step (1) with the same transfection steps as those of Example 2. Stable clones of recombinant CHO cells were obtained via screening under high pressure of puromycin (400 ng/ml).

(3) Recombinant CHO cells were seeded into 1.5 L proCHO5 medium containing 4 mmol/L glutamine, 0.68 mg/L hypoxanthine, 0.194 mg/L thymidine at 5×10⁵ cells/ml in 5 L shaking flasks and cultured at 37° C. for 72 hrs at a shaking speed of 110 r/min, then at 31° C. for another 216 hrs.

(4) The supernatant of the cell culture liquid at a culture volume of 1.5 L was harvested, and was filtrated through a 0.45 μm filter membrane at a volume of 500 ml. The protein of interest was purified using the heparin affinity chromatography column according to the method of Example 2, and identified by Western Blot. The results demonstrated that the FGFR2c extracellular fragment 146-356 could be t specifically recognized by the FGFR antibody.

### Example 4. Expressed of FGFR2c Extracellular Fragment 146-356 Polypeptide in Yeast Cells

1. Primer Design:

Upstream primer F3: 5′-ATAT CTCGAG GCCGCCACC ATG AACAAGAGAGCACCATAC-3′; the restriction site of Xho I is underlined;

Downstream primer R4: 5′-GCGC TCTAGA TCATTA CAGAACTGTCAACCATGC-3′; the restriction site of Xba I is underlined.

2. Vector Construction

The expression vector for Pichia yeasts, pPICZαA (Invitrogen), was used as the vector.

Step 2 of Example 2 was repeated using F5 and R5 as the primers and the restriction endonucleases Xho I and Xba 1. The resulting vectors were designated as pPICZαA-FGFR2c and pPICZαA-FGFR2c-S252W.

3. Transformation and Identification of Bacteria Cells

The plasmids pPICZαA-FGFR2c and pPICZαA-FGFR2c-S252W were transformed into the E. coli strain DH5α, respectively, following the method of transforming the E. coli strain DH5α using CaCl₂ as described in Example 1. Transformed DH5a was initially screened on LB plates containing the antibiotics Zeocin (100 μg/ml). The single colonies growing on the plate were picked and cultured at 37° C. for 16 hrs with shaking at 220 rpm. Plasmids were extracted at small scale and identified via restriction digestion (double digestion using Xho I and Xba I). Positive clones were sent to the sequencing company for sequencing.

4. Transformation of the Yeast Cell X33

After enzymatically linearized using Sac I (the digestion system was 10× buffer 2 μl, plasmid 10 μl, Sac I 1 μl, ddH₂O added to 20 μl), the purified plasmids were electrically transformed into competent cells of Pichia X33 with the specific steps as following: 80 μl X33 competent cells and 20 μl linearized plasmid were mixed together, transferred into a pre-cooled electroporation cuvette with a 0.2 cm gap, and incubated in an ice bath for 5 min; electroporation was conducted with electrical pulses at the voltage 1800 V and the duration 4.3 ms; immediately, 1 mL pre-cooled 1 M sorbitol was added to the cuvette; after gentle mix using a pipette, the mixture was transferred to a 1.5 mL EP tube; the cells were plated on a hypertonic complete medium (YPDS) plate containing 100 μg/ml Zeocin and cultured at 28° C. for 2-3 days.

5. Identification of recombinant strains and induction of expression. See the section “Expression of the fusion antibody ScFv-Fc” of the literation “Construction of a general expression vector for fusion antibody ScFv-Fc”. Dingding Wang et al., Chinese Journal of Bioengineering, 2011, 31(8): 110-117 for the specific steps.

6. Purification and Identification of the FGFR2c Extracellular Fragment 146-356 Protein

(1) Yeast cells were removed from the fermentation medium by centrifugation.

(2) The culture medium was concentrated using the 8000 KD column ultrafilter. The recombinant FGFR2c extracellular fragment 146-356 was preliminarily isolated and purified.

(3) The concentrate containing FGFR2c extracellular fragment 146-356 was further purified by affinity chromatography on a heparin column (same as that in Example 2), and identified by Western Blot. The results demonstrated that the FGFR2c extracellular fragment 146-356 could be specifically recognized by the FGFR antibody.

### Example 5. Expression of FGFR2c Extracellular Fragment 146-356 in Baculovirus-Infected Insect Cells

1. The vectors pFastBac-FGFR2c and pFastBac-FGFR2c-S252W were obtained following the method described in Example 3 (the primers were same as those described in Example 3, and the vector pFastBac was purchased from Invitrogen).

2. Transformation of Competent Cells of E. coli DH10Bac

Competent cells of E. coli DH10Bac (purchased from Invitrogen) were removed from the refrigerator at −80° C. and thawed on ice. 5 μL plasmid was sterilely added to competent cells of E. coli DH10Bac, and placed on ice for 30 min. After heat shock at 42° C. for 45 sec, the cells were plunged into ice for 2 min. After adding 0.9 ml SOC medium (Cat. No. 15544-034) at room temperature, the cells were shook at 225 rpm for 45 min at 37° C. The culture was diluted by adding SOC medium at a volume ratio of 1:10. 100 μL diluted culture was plated onto a LB plate containing 50 μg/ml kanamycin, 10 μg/ml tetracycline, 7 μg/ml gentamicin. 200 mg/ml IPTG and 20 mg/mL X-gal, and the plate was incubated at 37° C. for 24 hrs. The next day, white colonies were picked for culture in LB liquid medium containing 50 μg/ml kanamycin. The bacterial liquid was identified by PCR. The bacterial liquid identified to be correct was inoculated into 5 ml medium, and cultured at 37° C. overnight. Positive bacmids were extracted. Recombinant plasmid DNA was extracted using the Bac-to-bac HT Vector kit (purchased from Invitrogen Co., Cat. 1058-027). The results of transformation were detected by agarose electrophoresis.

3. See the literature “Expression of recombinant human soluble PDGFRβ/Fc in insect cells Sf9”. Qiuling Xie et al., Journal of Entomology, July 2009, 52(7): 743-748 for the specific transfection method of recombinant baculovirus vector pFastBac.

4. Expression of FGFR2c Extracellular Fragment

The supernatant and cells were collected by centrifugation at 16,000 rpm for 30 min at 4° C., and subjected to SDS-PAGE for identification by Western Blot. The results demonstrated that FGFR2c extracellular fragment 146-356 could be specifically recognized by the FGFR antibody.

### Example 6. Expression of the Fusion Protein of FGFR2c Extracellular Fragment 146-356-Fc Portion in Yeast Cells

1. Generation of the Gene Encoding the Fc Portion

(1) Primer Design

F9-Fc:
5′-CCCAAGAGCTGCGACAAGACCCACACCTGCCCCCCCTGTCCTGCTCC
AGAACTCCTGGGCGGACCCAGCGTGTTCCTGTTCCCCCCCAAGCCAAGGA
CACCCTG-3′;
F8-Fc:
5′-AAGCCCAAGGACACCCTGATGATCAGCAGGACCCCCGAGGTGACCTG
CGTGGTGGTGGACGTGAGCCACGAGGACCCACAGGTCAAGTTCAACTGGT
ACGTGGAC-3′;
F7-Pc:
5′-TTCAACTGGTACGTGGACGGCGTGCAGGTGCACAACGCCAAGACCAA
GCCCCGGGAGCAGCAGTACAACTCCACCTACAGAGTGGTGTCCGTGCTGA
CCGTGCTG-3′;
F6-Fc:
5′-TCCGTGCTGACCGTGCTGCACCAGAACTGGCTGGACGGCAAAGAGTA
CAAGTGCAAGGTCTCCAACAAGGCCCTGCCAGCCCCCATCGAGAAAACCA
TCAGCAAG-3′;
R6-Fc:
5′-CAGGGACACCTGGTTCTTGGTCATTTCCTCTCGAGAGGGTGGCAGGG
TGTACACCTGGGGTTCCCTGGGCTGGCCCTTGGCCTTGCTGATGGTTTTC
TC-3′;
R7-Fc:
5′-TCTTGTAGTTGTTCTCGGGCTGGCCGTTGCTCTCCCACTCCACGGCG
ATGTCGCTGGGGTAGAAGCCCTTCACCAGGCAGGTCAGGGACACCTGGTT
CTT-′3;
R8-Fc:
5′-CCCTGCTGCCATCTGCTCTTGTCCACGGTCAGCTTGCTGTACAGGAA
GAAGCTGCCGTCGCTGTCCAGCACTGGGGGGGTGGTCTTGTAGTTGTTCT
CGG-3′;
R9-Fc:
5′-CTTGCCGGGGGACAGGCTCAGGCTCTTCTGGGTGTAGTGGTTGTGCA
GGGCCTCGTGCATCACGCTGCAGCTGAACACGTTGCCCTGCTGCCATCTG
CTC-3′.

(2) The gene encoding the Fc portion was obtained by PCR with 4 steps. The first step was PCR using F6-Fc and R6-Fc as the primers. After 1% agarose gel electrophoresis, the band was recovered from the gel (TIANGEN, DP209). The second step was PCR using F7-Fc and R7-Fc as the primers and the PCR product of the first step as the template. The band was recovered from the gel. The third step was PCR using F8-Fc and R8-Fc as the primers and the PCR product of the second step as the template. The band was recovered from the gel. The fourth step was PCR using F9-Fc and R9-Fc as the primers and the PCR product of the third step as the template. The band was recovered from the gel.

The reaction system of PCR:

| | |
|---|---|
| PrimerSTAR max | 25 μL |
| Upstream primer | 3 μL |
| Downstream primer | 3 μL |
| Template | 4 μL |
| ddH₂O | 15 μL |
| Total volume: | 50 μL |

2. The genes encoding FGFR2c extracellular fragment, Fc portion and FGFR2c-L-Fc were amplified by PCR and overlapping PCR, respectively.

The FGFR2c template fragment was obtained by PCR using the pET3c-FGFR2c plasmid fragment gene as the template, and F10-FGFR2c and R10-FGFR2c as the primers. The Fc portion template fragment was obtained by PCR using the gene encoding the Fc portion as the template, and F11-Fc and R11-Fc as the primers.

(1) Primer Design, all in the Direction of 5′-3′

F10-FGFR2c: ATATAACAGT ATG GATGACGACGACAAG AACAAGAGAGCACCATAC; the restriction site of Spe I is underlined;

R10-FGFR2c: CGACCCACCACCGCCCGGAGCCACCGCCACC CAGAACTGTCAACCATGC;

F11-Fc: GGCGGTGGTGGGTCGGGTGGCGGCGGATCTCCCAAGAGCTGCGACAAG;

R11-Fc: ATATGAATTCCATTACTTGCCGCGGGACAGG; the restriction site of EcoR I is underlined;

(2) The Reaction System and the Reaction Conditions of PCR:

The reaction system of PCR

| | |
|---|---|
| PrimerSTAR max | 25 μL |
| F10 - FGFR2c/F11-Fc | 3 μL |
| R10 -FGFR2c/R11- Fc | 3 μL |
| Template | 4 μL |
| ddH₂O | 15 μL |
| total volume: | 50 μL |

The reaction conditions were: 96° C. 5 min; 31 cycles of 94° C. 15 sec, 60° C. 15 sec, 72° C. 5 sec; 72° C. 10 min.

(3) The DNA encoding FGFR2c and Fc portion was recovered following the method of recovering PCR products from the gel as described in Example 1.

The reaction system of overlapping PCR:

| | |
|---|---|
| PrimerSTAR max | 25 μL |
| FGFR2c template fragment | 2 μL |
| Fc template fragment | 2 μL |
| ddH₂O | 15 μL |
| total volume: | 44 μL |

The reaction conditions were: 96° C. 5 min; 5 cycles of 94° C. 15 sec, 60° C. 15 sec, 72° C. 5 sec; 72° C. 10 min.

(4) After the above 5 cycles were completed, the primers F10-FGFR2c and R11-Fc were added, 3 μL each. Then, additional 30 cycles were run. The PCR product was recovered from the gel following the method as described in Example 1 to obtain the FGFR2c-L-Fc gene.

3. The FGFR2c-L-Fc gene was ligated into the pPICZαA expression vector following the method as described in Example 4. The results of identification by double digestion and sequencing demonstrated that the FGFR2c-L-Fc-pPICZαA recombinant plasmid was successfully constructed.

4. The fusion protein FGFR2b-L-Fc was expressed by induction, purified and identified following the method as described in Example 4. The results demonstrated that the fusion protein FGFR2b-L-Fc could be specifically recognized by the FGFR antibody.

### Example 7. Comparison of Stability

S252W mutant msFGFR2c (147-366) and wild type FGFR2c (147-366) were prepared following the method described in the literature (Prokaryotic expression of wild type and mutant FGFR2IIIc extracellular fragments and their inhibitory effect on tumor. Xueting Liu, Jinan University Master Theses, 2008: 8). S252W mutant msFGFR2c (151-377) and wild type FGFR2c (151-377) were prepared following the method described in the literature (Prokaryotic expression of FGFR2IIIc extracellular fragment and its inhibitory effect on proliferation of prostate cancer cells. Ying He, Ju Wang et al., Chinese Journal of Bioengineering, 2009, 29 (7): 7-11).

1. Detection of Stability of sFGFR2c by SDS-PAGE

S252W mutant msFGFR2c polypeptide (prepared in Example 1) and wild type wsFGFR2c polypeptide (prepared in Example 1), S252W mutant msFGFR2c (147-366), wild type FGFR2c (147-366), S252W mutant msFGFR2c (151-377), wild type FGFR2c (151-377) were dialyzed against 25 mM HEPES buffer for 12 h. respectively. Then, they were filtered through a 0.22 μm filter membrane, and diluted with 25 mM HEPES buffer to a protein concentration of 100 μg/ml. For each of them, an aliquot of 2 ml was stored in a refrigerator at 4° C. Then, a sample of 160 μl supernatant was taken at the same time in Day 1, 3, 5, and 7 for each of them (for each sampling, the samples were centrifuged at 16,000 rpm for 15 min at 4° C., and the pellets were discarded). The samples were stored at −70° C. Then, the stability of the proteins was detected by SDS-PAGE. Meanwhile, a protein solution which was dialyzed and filtered through a membrane was used as the control.

2. Results

The results are shown in FIG. 3. FIG. 3A is an electrophoretogram showing the stability of FGFR2c extracellular fragment 146-356 prepared in the present invention. Lane 1 is S252W mutant msFGFR2c used as a control. Lanes 2, 3, 4, and 5 are S252W mutant msFGFR2c samples of Days 1, 3, 5, and 7, respectively. Lane 6 is wild type wsFGFR2c used as a control. Lanes 7, 8, 9, and 10 are wild type wsFGFR2c samples of Days 1, 3, 5, and 7, respectively. As can be seen from the figure, the FGFR2 extracellular fragment prepared in the present invention had substantially no degradation. The protein was relatively stable.

FIG. 3B is an electrophoretogram showing the stability of S252W mutant msFGFR2c (147-366aa) and wild type FGFR2c (147-366aa) prepared following the literature. Lane 1 is S252W mutant msFGFR2c (147-366) used as a control. Lanes 2, 3, 4, and 5 are S252W mutant msFGFR2c (147-366) samples of Days 1, 3, 5, and 7, respectively. Lane 6 is wild type FGFR2c (147-366) used as a control. Lanes 7, 8, 9, and 10 are wild type FGFR2c (147-366) samples of Days 1, 3, 5, and 7, respectively. As can be seen from the figure, wild type and S252W mutant had litter degradation on Days 1 and 3 (Lanes 2, 3, 7, and 8), further degradation on Day 5 (Lanes 4 and 9), and substantially complete degradation on Day 7 (Lanes 5 and 10).

FIG. 3C is an electrophoretogram showing the stability of S252W mutant msFGFR2c (151-377aa) and wild type FGFR2c (151-377aa) prepared following the literature. Lane 1 is S252W mutant msFGFR2c (151-377aa) used as a control. Lanes 2, 3, 4, and 5 are S252W mutant msFGFR2c (151-377aa) samples of Days 1, 3, 5, and 7, respectively. Lane 6 is wild type FGFR2c (151-377aa) used as a control. Lanes 7, 8, 9, and 10 are wild type FGFR2c (151-377aa) samples of Days 1, 3, 5, and 7, respectively. As can be seen from the figure, wild type and S252W mutant had degradation on Days 1 and 3 (Lanes 2, 3, 7, and 8), further degradation on Day 5 (Lanes 4 and 9), and substantially complete degradation on Day 7 (Lanes 5 and 10).

Conclusion: FGFR2c extracellular fragment 146-356 constructed in the present invention is more stable than FGFR2c extracellular fragment (147-366 aa), FGFR2c extracellular fragment (151-377 aa) in the prior art, which is helpful in improving the process yield.

### Example 8. FGFR2c Extracellular Fragment 146-356 Inhibits Malignant Tumors by Inhibiting EGF Signaling in DU145 Cells

1. Cell Culture

DU145 prostate cancer cells (from ATCC) were subcultured in a 50 cm² cell culture flask (purchased from Thermo) filled with 1640 medium containing 10% (v/v) fetal calf serum in a cell incubator at 37° C., 5% CO₂. During culture, passaged could be conducted when the cells grew to about 80% confluen. Cells were digested with 0.25% w/v trypsin solution. The cell density was at least 5×10⁵ cells/mL for normal passage.

2. Co-Immunoprecipitation (Co-IP) Detection of Binding of sFGFR2c to Exogenous and Endogenous EGFR

1) Human Embryonic Kidney Cells, 293T Cells (ATCC, CRL-3216) Overexpressing EGFR

Transfection and Induction:

(1) Transfection: the specific method was same as the Lipofectamine™ 2000 liposomal transfection as described in Example 2.

(2) Induction: After culture for 24 hrs, the residual medium was removed by washing with 1×PBS and replaced with DMEM starvation media containing 0.5% (v/v) FBS. Induction was conducted 12 hours later. Specific steps: EGF (20 ng/ml) or FGF-2 (20 ng/ml) or wsFGFR2c (1 μg/ml, prepared in Example 1) or msFGFR2c (1 μg/ml, prepared in Example 1) in DMEM medium containing 3% (v/v) FBS was added to a 6-well plate, 2 ml per well. The first well received DMEM medium containing 3% (v/v) FBS only; the second well received DMEM medium containing 3% (v/v) FBS+wsFGFR2c; the third well received DMEM medium containing 3% (v/v) FBS+wsFGFR2c+EGF; the fourth well received DMEM medium containing 3% (v/v) FBS+wsFGFR2c+FGF-2; the fifth well received DMEM medium containing 3% (v/v) FBS+msFGFR2c; the sixth well received DMEM medium containing 3% (v/v) FBS+msFGFR2c+EGF; the seventh well received DMEM medium containing 3% (v/v) FBS+msFGFR2c+FGF-2.

2) Co-IP Sample Preparation

(1) Sample Preparation by Cell Lysis

\textcircled{1} After induction for 1 hr, the cells were first washed twice with ice pre-cooled 1×PBS. Then, the 1×PBS was aspirated.

\textcircled{2} The cell lysis solution (Beyotime, Cat. P0013) was added to the cells of step \textcircled{1} at 400 μL per well.

\textcircled{3} All the cells were scraped off using a cell scraper and collected into a EP tube.

\textcircled{4} The cells were vortexed for 30 sec, and placed on ice for 10 min for lysis. Then, the cells were centrifuged at 16,000 rpm for 10 min at 4° C. The pellet was discarded, and the supernatant was collected. Thus, a protein sample of cell lysate was obtained.

(2) Magnetic Bead Incubation, Sample Preparation

\textcircled{1} Magnetic beads (dynabeads M-270 streptavidin) were added into EP tubes at 40 μL/tube, and washed three times with 600 μL 1×PBS.

\textcircled{2} PBS was removed. A protein sample of cell lysate of 300 μL was added to each tube, and incubated with magnetic beads for 1 hr.

\textcircled{3} After incubation was completed, the magnetic beads were washed with PBS containing 0.05% (v/v) Tween. The washing solution was replaced every 5 min for 8 times. Afterwards, the magnetic beads were washed twice with PBS, 5 min each.

\textcircled{4} PBS was aspirated, and 5×SDS loading buffer was added. The tubes were kept in a boiling water bath for 5 min. Thus, samples for Western blot were prepared.

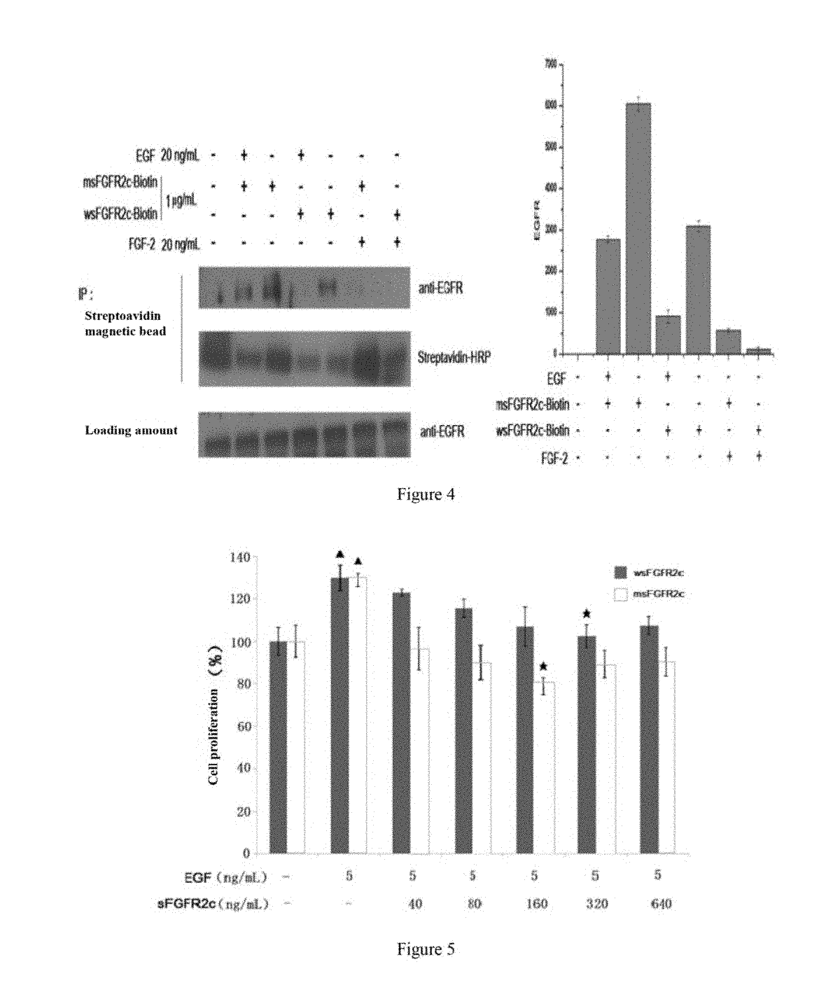

\textcircled{5} Detection was conducted by Western blot. The results are shown in FIG. 4.

3. Detection of Cell Proliferation by CCK8 Assay

(1) Plating: When growing to 70-80% confluen after passage. DU145 cells were digested with trypsin. 1640 medium containing 10% (v/v) FBS was added to dilute the cells to 3×10⁴ cells/mL cell suspension. Then, cells were added to a 96-well cell culture plate at the amount of 4-5×10³ cells/well, i.e., 150 μL cell suspension per well, and cultured in an incubator at 37° C., 5% CO₂ for 24 hrs.

(2) Starving: The medium was aspirated and 1640 medium containing 0.1% active carbon treated serum was added at 150 μL/well. The cells were starved for 24 hrs.

(3) Inducing: The starvation medium was aspirated and 1640 medium containing 0.1% active carbon treated serum was added again. Furthermore, sFGFR2c was added at 150 μL/well for induction. The induction process: for 7 columns on a 96-well plate, Column 1 received 150 μl medium only; Column 2 received medium+EGF; Column 3 received medium+EGF+sFGFR2c (40 ng/ml); Column 4 received medium+EGF+sFGFR2c (80 ng/ml); Column 5 received medium+EGF+sFGFR2c (160 ng/ml); Column 6 received medium+EGF+sFGFR2c (320 ng/ml); Column 7 received medium+EGF+sFGFR2c (640 ng/ml); culture for 48 hrs for induction.

(4) Reading: the CCK8 working solution was first prepared following the CCK8 instructions. Then, the medium solution in each well was aspirated. The CCK8 working solution was added at 110 μL/well. The plate was incubated in an incubator at 37° C. for 4 hrs. Then, the plate was removed and gently shaked. The absorbance at two wavelengths 450/570 nm was read on a microplate reader.

4. Experimental Results

(1) Binding of EGFR to sFGFR2c as Detected by Co-IP (See FIG. 4)

The ability of EGFR endogenously expressed by DU145 cells to bind to sFGFR2c was detected by Co-IP assay. It was found that in the presence of EGF, msFGFR2c (i.e., S252W mutant msFGFR2c, the same below) had an impaired ability to bind to EGFR (see FIG. 3B). Likewise, the same applies to wsFGFR2c (i.e., wild type wsFGFR2c). In addition, we also found that induction of DU145 cells by simultaneously adding FGF-2 and sFGFR2 could also lead to binding between EGFR and sFGFR2c but at a much lower level than that induced by sFGFR2c alone or in combination with EGF.

(2) EGF induced proliferation of DU145 cells. If sFGFR2c was simultaneously added for induction, it had inhibitory effect on proliferation of DU145 (see FIG. 5). When EGF was at a concentration of 5 ng/mL, the inhibitory effect on EGF-induced proliferation became more evident with the increasing concentration of sFGFR2c. Especially for msFGFR2c, the inhibitory effect reached the top at the concentration of 160 ng/mL, an inhibition rate of 21.1% as relative to the control group. When the concentration of msFGFR2c continued to increase, the inhibitory effect decreased. The inhibitory effect of wild type wsFGFR2c was relatively weak, a growth rate of 102.5% as relative to the control group at 320 ng/mL. Likewise, the inhibitory effect decreased as the concentration for induction continued to increase.

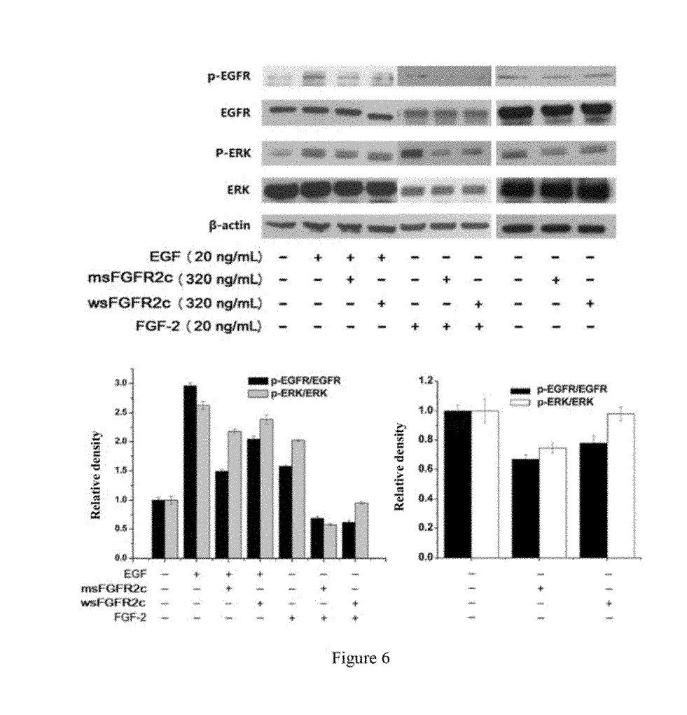

(3) sFGFR2c Inhibited EGFR/ERK Signaling Pathway (See FIG. 6)

As detected by Western blot, it was found that after DU145 cells were induced with sFGFR2c, sFGFR2c could inhibit EGF induced EGFR and ERK phosphorylation (see FIG. 6); and that when co-induced with EGF, both of them could inhibit EGFR and ERK activation and msFGFR2c had a stronger inhibitory effect than wsFGFR2c. Similarly, sFGFR2c could also inhibit FGF-2 induced ERK phosphorylation.

(4) sFGFR2c Inhibited FGF Signaling Pathway

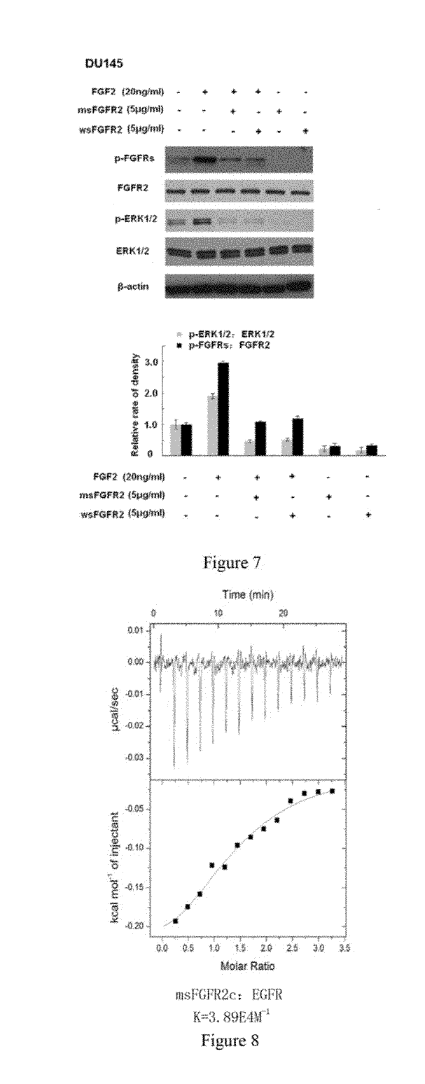

As detected by Western blot, it was found that after DU145 cells were induced with sFGFR2c, sFGFR2c could inhibit FGFRs and ERK phosphorylation (see FIG. 7); and that when co-induced with FGF-2, both of them could inhibit FGFRs and ERK activation and msFGFR2c had a stronger inhibitory effect than wsFGFR2c.

### Example 9. Detection of the Interaction Between FGFR2c Extracellular Fragment 146-356 with EGFR Using the Isothermal Titration Calorimetry (iTC)

This example describes the experiment of binding of msFGFR2c prepared following the method as described in Example 1 with EGFR. The experimental results obtained are shown in FIG. 8. msFGFR2c and EGFR extracellular fragment prepared in-house were dialyzed against the isothermal Titration Calorimetry (iTC) buffer (25 mM HEPES, 0.15 mM NaCl, 5% glycerol, pH=7.5), respectively. After dialysis was completed, the proteins were centrifuged at 18,000 rpm for 30 min at 4° C. for subsequent ITC experiments. 200 μl EGFR was added to the sample cell at the sample concentration of 20 μmol/L. Then 40 μL msFGFR2c at 150 μmol/L was added to the loading syringe for titration. The reaction conditions were: first titration, 0.4 μL; each of rest titrations, 3 μL; one titration every 2 min; 14 titrations in total.

The results demonstrated that msFGFR2c is capable of interacting with EGFR and has good binding effect.

The above examples are merely provided to describe several embodiments of the present invention in a relatively specific and detailed way, but cannot thereby be construed as limiting the scope of the present invention. It should be noted that some modifications and improvements will be apparent to those of ordinary skill in the art without departing from the concept of the present invention, which all fall into the scope of the present invention. Accordingly, the scope of the present invention is defined in the appended claims.

1. An isolated protein comprising the amino acid sequence as set forth in SEQ ID NO: 1 or 2.

2. An isolated protein comprising the following amino acid sequence and having the function of inhibiting EGF signaling but not activating FGF signaling:
i) an amino acid sequence obtained from the amino acid sequence as set forth in SEQ ID NO: 1 or 2 by deleting, substituting, inserting, and/or adding one or more amino acids;
ii) an amino acid sequence having a homology of 80% or more with the amino acid sequence as set forth in SEQ ID NO: 1 or 2; or
iii) an amino acid sequence encoded by a nucleic acid which hybridizes under stringent conditions to the complementary strand of the nucleic acid encoding the amino acid sequence as set forth in SEQ ID NO: 1 or 2.

3. The isolated protein according to claim 2, wherein the isolated protein has a human origin.

4. An isolated nucleic acid encoding the isolated protein of claim 1.

5. A vector comprising the nucleic acid of claim 4.

6. A host cell comprising the vector of claim 5.

7. The host cell according to claim 6, wherein said host cell is any one of CHO cells, E. coli cells, insect cells, and yeast cells.

8. A fusion protein of the isolated protein of claim 1 fused with another polypeptide.

9. The fusion protein according to claim 8, wherein the isolated protein is fused with an epitope tag sequence of a human immunoglobulin or the Fc portion of a human immunoglobulin.

10-11. (canceled)

12. An isolated nucleic acid encoding the isolated protein of claim 2.

13. A vector comprising the nucleic acid of claim 12.

14. A host cell comprising the vector of claim 13.

15. The host cell according to claim 14, wherein said host cell is any one of CHO cells, E. coli cells, insect cells, and yeast cells.

16. A fusion protein of the isolated protein of claim 2 fused with another polypeptide.

17. The fusion protein according to claim 16, wherein the isolated protein is fused with an epitope tag sequence of a human immunoglobulin or the Fc portion of a human immunoglobulin.

18. A method for treating a malignant tumor in an animal comprising administering the isolated protein of claim 1 to the animal.

19. A method for treating a malignant tumor in an animal comprising administering the isolated protein of claim 2 to the animal.

20. A pharmaceutical composition comprising the isolated protein of claim 1 and a pharmaceutically acceptable carrier.

21. A pharmaceutical composition comprising the isolated protein of claim 2 and a pharmaceutically acceptable carrier.


SEQUENCE LISTING (26 sequences)

SEQ ID NO: 1 - protein, 211 aa, Homo sapiens
NKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVL

SEQ ID NO: 2 - protein, 211 aa, artificial sequence
  amino acid sequence of S252W mutant msFGFR2c
NKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERWPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVL

SEQ ID NO: 3 - DNA, 633 bp, Homo sapiens
aacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctg

SEQ ID NO: 4 - DNA, 633 bp, artificial sequence
  nucleotide sequence encoding S252W mutant msFGFR2c
aacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatggcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctg

SEQ ID NO: 5 - DNA, 26 bp, artificial sequence
  primer F1
cgcatatgaacaagagagcaccatac

SEQ ID NO: 6 - DNA, 32 bp, artificial sequence
  primer R1
atggatccctattacagaactgtcaaccatgc

SEQ ID NO: 7 - DNA, 28 bp, artificial sequence
  primer F2
ttgtggagcgatggcctcaccggcccat

SEQ ID NO: 8 - DNA, 28 bp, artificial sequence
  primer R2
atgggccggtgaggccatcgctccacaa

SEQ ID NO: 9 - DNA, 40 bp, artificial sequence
  primer F3
atatggatccgccgccaccatgaacaagagagcaccatac

SEQ ID NO: 10 - DNA, 34 bp, artificial sequence
  primer R3
gcgcaagctttcattacagaactgtcaaccatgc

SEQ ID NO: 11 - DNA, 40 bp, artificial sequence
  primer F4
atatgctagcgccgccaccatgaacaagagagcaccatac

SEQ ID NO: 12 - DNA, 34 bp, artificial sequence
  primer R4
gcgcgaattctcattacagaactgtcaaccatgc

SEQ ID NO: 13 - DNA, 40 bp, artificial sequence
  primer F5
atatctcgaggccgccaccatgaacaagagagcaccatac

SEQ ID NO: 14 - DNA, 34 bp, artificial sequence
  primer R5
gcgctctagatcattacagaactgtcaaccatgc

SEQ ID NO: 15 - DNA, 105 bp, artificial sequence
  primer F9-Fc
cccaagagctgcgacaagacccacacctgccccccctgtcctgctccagaactcctgggcggacccagcgtgttcctgttcccccccaagcccaaggacaccctg

SEQ ID NO: 16 - DNA, 105 bp, artificial sequence
  primer F8-Fc
aagcccaaggacaccctgatgatcagcaggacccccgaggtgacctgcgtggtggtggacgtgagccacgaggacccacaggtcaagttcaactggtacgtggac

SEQ ID NO: 17 - DNA, 105 bp, artificial sequence
  primer F7-Fc
ttcaactggtacgtggacggcgtgcaggtgcacaacgccaagaccaagccccgggagcagcagtacaactccacctacagagtggtgtccgtgctgaccgtgctg

SEQ ID NO: 18 - DNA, 105 bp, artificial sequence
  primer F6-Fc
tccgtgctgaccgtgctgcaccagaactggctggacggcaaagagtacaagtgcaaggtctccaacaaggccctgccagcccccatcgagaaaaccatcagcaag

SEQ ID NO: 19 - DNA, 99 bp, artificial sequence
  primer R6-Fc
cagggacacctggttcttggtcatttcctctcgagagggtggcagggtgtacacctggggttccctgggctggcccttggccttgctgatggttttctc

SEQ ID NO: 20 - DNA, 100 bp, artificial sequence
  primer R7-Fc
tcttgtagttgttctcgggctggccgttgctctcccactccacggcgatgtcgctggggtagaagcccttcaccaggcaggtcagggacacctggttctt

SEQ ID NO: 21 - DNA, 100 bp, artificial sequence
  primer R8-Fc
ccctgctgccatctgctcttgtccacggtcagcttgctgtacaggaagaagctgccgtcgctgtccagcactgggggggtggtcttgtagttgttctcgg

SEQ ID NO: 22 - DNA, 100 bp, artificial sequence
  primer R9-Fc
cttgccgggggacaggctcaggctcttctgggtgtagtggttgtgcagggcctcgtgcatcacgctgcagctgaacacgttgccctgctgccatctgctc

SEQ ID NO: 23 - DNA, 40 bp, artificial sequence; identical to SEQ ID NO: 13
  primer F5-FGFR2c

SEQ ID NO: 24 - DNA, 48 bp, artificial sequence
  primer R10-FGFR2c
cgacccaccaccgccggagccaccgccacccagaactgtcaaccatgc

SEQ ID NO: 25 - DNA, 48 bp, artificial sequence
  primer F11-Fc
ggcggtggtgggtcgggtggcggcggatctcccaagagctgcgacaag

SEQ ID NO: 26 - DNA, 32 bp, artificial sequence
  primer R11-Fc
atattctagatcattacttgccgggggacagg